SEQUENCE LISTING (9 sequences)

SEQ ID NO: 1 - DNA, 133 bp, Artificial Sequence
  PTM24 binding domain
tcacgctcaggattcacttgcccccaattatcatcctaagcagaagtgtatattcttatttgtaaagattctattaactcatttgattcaaaatatttaaaatacttcctgtttcacctactctgctatgcac

SEQ ID NO: 2 - DNA, 71 bp, Artificial Sequence
  PTM24 spacer, branchpoint and polypyrimidine tract
ccgcggaacattattataacgttgctcgaatactaactggtacctcttcttttttttttgatatcctgcag

SEQ ID NO: 3 - DNA, 141 bp, Artificial Sequence
  PTM15 trans-splicing domain
ataaaacccatcattatagctcattatcaaatcacgctcaggattcacttgcctccaattatcatcctaaccgcggaacattattataacgttgctcgaatactaactggtacctcttcttttttttttgatatcctgcag

SEQ ID NO: 4 - DNA, 204 bp, Artificial Sequence
  PTM24 trans-splicing domain
tcacgctcaggattcacttgcccccaattatcatcctaagcagaagtgtatattcttatttgtaaagattctattaactcatttgattcaaaatatttaaaatacttcctgtttcacctactctgctatgcacccgcggaacattattataacgttgctcgaatactaactggtacctcttcttttttttttgatatcctgcag

SEQ ID NO: 5 - RNA, 8 nt, Artificial Sequence
  PTM 5′ splice site consensus sequence
agguragu

SEQ ID NO: 6 - RNA, 7 nt, Artificial Sequence
  PTM 3′ branch point consensus sequence
ynyurac

SEQ ID NO: 7 - DNA, 258 bp, Artificial Sequence
  PTM intron 5′ fragment sequence
gtagttcttttgttcttcactattaagaacttaatttggtgtccatgtctctttttttttctagtttgtagtgctggaaggtatttttggagaaattcttacatgagcattaggagaatgtatgggtgtagtgtcttgtataatagaaattgttccactgataatttactctagttttttatttcctcatattattttcagtggctttttcttccacatctttatattttgcaccacattcaacactgtagcggccgc

SEQ ID NO: 8 - DNA, 270 bp, Artificial Sequence
  PTM intron 3′ fragment sequence
ccaactatctgaatcatgtgccccttctctgtgaacctctatcataatacttgtcacactgtattgtaattgtctcttttactttcccttgtatcttttgtgcatagcagagtacctgaaacaggaagtattttaaatattttgaatcaaatgagttaatagaatctttacaaataagaatatacacttctgcttaggatgataattggaggcaagtgaatcctgagcgtgatttgataatgacctaataatgatgggttttatttccag

SEQ ID NO: 9 - RNA, 18 nt, Artificial Sequence
  PTM ISAR sequence
gggcugauuuuuccaugu

(19) United States
(12) Patent Application Publication
(10) Pub. No.: US 2004/0180429 A1
(43) Pub. Date: Sep. 16, 2004

(54) Spliceosome mediated RNA trans-splicing in stem cells

(76) Inventors: Lloyd G. Mitchell, Bethesda, MD (US); John Englehardt, Iowa City, IA (US); Xiao Ming Liu, Iowa City, IA (US)

Correspondence Address: BAKER & BOTTS, 30 ROCKEFELLER PLAZA, NEW YORK, NY, 10112

(21) Appl. No.: 10/456,273

(22) Filed: Jun. 5, 2003

Related U.S. Application Data
(60) Provisional application No. 60/386,594, filed on Jun. 5, 2002.

(51) Int. Cl. C12N 5/08

(52) U.S. Cl. 435/366; 435/320.1

(57) ABSTRACT

The present invention provides methods and compositions for generating novel nucleic acid molecules through targeted spliceosomal mediated trans-splicing in stem cells. The compositions of the invention include stem cells engineered to express pre-trans-splicing molecules (PTMs) designed to interact with a target precursor messenger RNA molecule (target pre-mRNA) and mediate a trans-splicing reaction resulting in the generation of novel chimeric RNA molecules (chimeric RNA). In particular, the stem cells of the present invention are genetically engineered to express a PTM that will interact with a specific target pre-mRNA expressed within a stem cell as it differentiates so as to result in correction of a genetic defect responsible for a genetic disorder. The methods of the invention encompass transferring a nucleic acid molecule capable of encoding a PTM of interest into a stem cell followed by transplantation of the PTM modified stem cell into a host. As the stem cell differentiates the target pre-mRNA is expressed thereby providing the substrate for a trans-splicing reaction. The present invention is based on the successful transfer and expression of a nucleic acid molecule encoding a PTM capable of interacting with a cystic fibrosis transmembrane conductance regulator (CFTR) pre-mRNA into primary human surface airway progenitor cells. The methods and compositions of the present invention can be used to correct genetic defects associated with a variety of different disorders such as cystic fibrosis, hemophilia, sickle cell anemia, Tay-Sachs disease, thalassemias, polycystic kidney disease and muscular dystrophy, to name a few.

### 1. INTRODUCTION

The present invention provides methods and compositions for generating novel nucleic acid molecules through targeted spliceosomal mediated trans-splicing in stem cells. The compositions of the invention include stem cells engineered to express pre-trans-splicing molecules (PTMs) designed to interact with a target precursor messenger RNA molecule (target pre-mRNA) and mediate a trans-splicing reaction resulting in the generation of novel chimeric RNA molecules (chimeric RNA). In particular, the stem cells of the present invention are genetically engineered to express a PTM that will interact with a specific target pre-mRNA expressed within a stem cell as it differentiates so as to result in correction of a genetic defect responsible for a genetic disorder. The methods of the invention encompass transferring a nucleic acid molecule capable of encoding a PTM of interest into a stem cell followed by transplantation of the PTM modified stem cell into a host. As the stem cell differentiates the target pre-mRNA is expressed thereby providing the substrate for a trans-splicing reaction. The present invention is based on the successful transfer and expression of a nucleic acid molecule encoding a PTM capable of interacting with a cystic fibrosis transmembrane conductance regulator (CFTR) pre-mRNA into primary human surface airway progenitor cells. The methods and compositions of the present invention can be used to correct genetic defects associated with a variety of different disorders such as cystic fibrosis, hemophilia, sickle cell anemia, Tay-Sachs disease, thalassemias, polycystic kidney disease and muscular dystrophy, to name a few.

### 2. BACKGROUND OF THE INVENTION

Recent advances in molecular biology, including the sequencing of the human genome has provided valuable information concerning the genetic basis of disease. Gene therapy is based on the premise that inherited genetic disorders can be corrected at the level of nucleic acid molecules. Challenges associated with the use of gene therapy include the development of effective approaches for delivering genetic material to the appropriate cells of the patient in a manner that is safe, specific and efficient.

Until recently, the practical application of targeted trans-splicing to modify specific target genes has been limited to group I ribozyme-based mechanisms. Using the Tetrahymena group I ribozyme, targeted trans-splicing was demonstrated in E. coli. coli (Sullenger B. A. and Cech. T. R., 1994, Nature 341:619-622), in mouse fibroblasts (Jones, J. T. et al., 1996, Nature Medicine 2:643-648), human fibroblasts (Phylacton, L. A. et al. Nature Genetics 18:378-381) and human erythroid precursors (Lan et al., 1998, Science 280:1593-1596). While several applications of targeted RNA trans-splicing driven by modified group I ribozymes have been explored, targeted trans-splicing mediated by native mammalian splicing machinery, i.e., spliceosomes, appears to be a more promising approach.

Spliceosomal mediated trans-splicing utilizes the cellular splicing machinery to repair inherited genetic defects at the RNA level by replacing mutant exons. The use of such techniques has a number of advantages associated with their use. For example, the repaired product is always under endogenous control and correction will only occur in cells endogenously expressing the target pre-mRNA. In addition, genetic diseases can be corrected regardless of the mode of inheritance. Finally, the use of trans-splicing reduces the size of the corrective insert into an expression vector.

U.S. Pat. Nos. 6,083,702, 6,013,487 and 6,280,978 describe the use of PTMs to mediate a trans-splicing reaction by contacting a target precursor mRNA to generate novel chimeric RNAs. The present invention provides methods and compositions for use of specific PTM molecules designed to correct specific defective genes in stem cells. The specific PTMs of the invention may be used to treat a variety of different genetic disorders. Since a trans-splicing reaction will only occur in differentiating stem cells expressing the target pre-mRNA, the present invention avoids the problems associated with expression of deleterious genes in stem cells.

### 3. SUMMARY OF THE INVENTION

The present invention relates to compositions and methods for expressing novel nucleic acid molecules through spliceosome-mediated targeted trans-splicing in stem cells. In particular, the compositions of the invention include stem cells engineered to express pre-trans-splicing molecules (hereinafter referred to as “PTM's”) designed to interact with a specific target pre-mRNA molecule (hereinafter referred to as “target pre-mRNA”) and mediate a spliceosomal trans-splicing reaction resulting in the generation of a novel chimeric RNA molecule (hereinafter referred to as “chimeric RNA”). The invention is based on the successful targeted trans-splicing of the cystic fibrosis transmembrane conductance regulator (CFTR) target pre-mRNA in primary human surface airway progenitor cells.

The compositions of the invention include stem cells, or progenitor cells, engineered to express PTMs designed to interact with a specific target pre-mRNA molecule and mediate a spliceosomal trans-splicing reaction resulting in the generation of a novel chimeric RNA molecule. Such PTMs are designed to correct genetic defects in the specific target pre-mRNA. Since a trans-splicing reaction will only occur in differentiated stem cells expressing the target pre-mRNA the present invention provides methods for targeting gene therapy without the problems associated with deleterious gene expression in stem cells. The general design, construction and genetic engineering of PTMs and demonstration of their ability to successful mediate trans-splicing reactions within the cell are described in detail in U.S. Pat. Nos. 6,083,702, 6,013,487 and 6,280,978 as well as patent Ser. Nos. 09/756,095, 09/756,096, 09/756,097 and 09/941,492, the disclosures of which are incorporated by reference in their entirety herein.

The methods of the invention encompass contacting a stem cell or progenitor cell with a nucleic acid molecule capable of encoding a PTM wherein said PTM is designed to interact with a specific target pre-mRNA under conditions in which a portion of the PTM is spliced to the target pre-mRNA to form a novel chimeric RNA that results in correction of a specific genetic defect. Nucleic acid molecules encoding PTMs may be transferred into a target stem cell in vivo or ex vivo followed by expression of the nucleic acid molecule to form a PTM capable of mediating a trans-splicing reaction. If genetically engineered ex vivo, the stem cells are then transplanted into the subject host. The PTMs of the invention are genetically engineered so that the novel chimeric RNA resulting from the trans-splicing reaction encodes a protein that complements a missing defective or inactive protein within the cell. Alternatively, the PTMs of the invention are genetically engineered so that the novel chimeric RNA resulting from the trans-splicing reaction encodes a protein that may be useful for imaging gene expression. The methods and compositions of the invention can be used in gene repair for the treatment of various genetic disorders, such as cystic fibrosis, hemophilia, sickle cell anemia, Tay-Sachs disease, thalassemias, polycystic kidney disease and muscular dystrophy to name a few.

### 4. BRIEF DESCRIPTION OF THE DRAWINGS

FIG. 1. Schematic representation of different trans-splicing reactions. (a) trans-splicing reactions between the target 5‘splice site and PTM’s 3′ splice site, (b) trans-splicing reactions between the target 3‘splice site and PTM’s 5′ splice site and (c) replacement of an internal exon by a double trans-splicing reaction in which the PTM carries both 3′ and 5′ splice sites. BD, binding domain; BP, branch point sequence; PPT, polypyrimidine tract; and ss, splice sites.

FIG. 2. Model of trans-splicing mediated mRNA repair of genomic A F508 CFTR using PTM 24.

FIG. 3A. Schematic diagram of PTM 24 construct.

FIG. 3B. Complete sequence of the trans-splicing domain of PTM15 and PTM24.

FIG. 4. Schematic representation of the protocol used to assess CFTR stem cell reconstitution in airway stem cells. First, primary airway epithelial cells are transduced with either LacZ or CFTR expressing retroviruses. Assessment of transgene expression is performed by Southern blot or X-gal staining (Usually 10-20%). Primary cells are then seeded into human bronchial xenografts and allowed to reconstitute for 5-6 weeks. Xenograft airway epithelium is then assessed for CFTR or LacZ transgene expression.

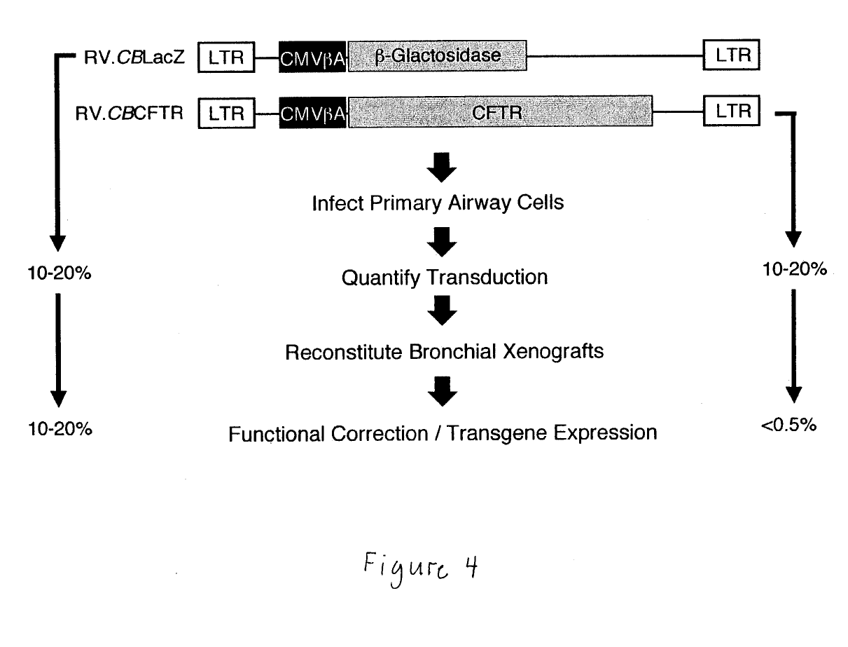

FIG. 5. Transgene Expression Analysis by In Situ Hybridization. To directly compare transgene expression from xenografts infected with CFTR or LacZ expressing retroviruses, an in situ expression assay was developed using a RNA probe complementary to the 3′-untranslated region of both vector transgenes. The probe is referred to as the “Universal Probe” and its position is indicated in Panel C. To test the sensitivity of this probe, serial sections from RV.CBLacZ reconstituted xenografts were used to detect expression of the transgene by in situ hybridization (A) or X-gal histochemical staining (B). Results demonstrate a complete concordance in clone location and sensitivity, indicating that such detection schemes can be used for comparing CFTR to LacZ gene expression. In Panels D and E, xenografts reconstituted with either RV.CBCFTR (D) or RV.CBLacZ (E) retroviruses were sectioned and probed with antisense Universal Probe to detect transgene expression. As seen, many more clones exist in the surface airway epithelium in RV.CBLacZ infected xenografts as compared to RV.CBCFTR. White grains indicated expression of mRNA target. Quantification of these results is given in FIG. 7.

FIG. 6. CFTR Transgene Expression in Infrequent Non-ciliated Clones. Despite the low abundance of CFTR expression from the retroviral vector RV.CBCFTR that expresses the full-length CFTR cDNA, expression of both CFTR transgene derived mRNA and CFTR protein could be detected in infrequent non-ciliated cell clones. (A) In situ hybridization using the antisense universal probe to the 3′ untranslated region of the retrovirally derived CFTR transgene. One clone expressing CFTR mRNA is boxed in panel A. (B) A serial section from that shown in panel A was stained for CFTR protein. Both the serial in situ staining and CFTR protein staining are shown (top and bottom panel of B). The middle panel of B is a Nomarski photomicrograph of the same field in the bottom panel. The region of CFTR expression is confined to undifferentiated non-ciliated cells. This demonstrates that ectopic, unregulated, expression of CFTR in airway stem cells affects their capacity to proliferate and differentiate cells and implies that high level CFTR expression may be toxic to airway progenitor cells.

FIG. 7. LacZ and CFTR expression in surface airway epithelial (BAE) cells of xenografts using conventional retroviral vectors. The data represents transgene expression in primary airway epithelia following infection in vitro and the level of sustained expression following reconstitution of airway epithelia in xenografts in vivo.

FIG. 8. Retroviral vectors designed to assay PTM delivery of CFTR. Three retroviral vectors were compared for their ability to reconstitute transgene expression in airway stem cells by xenograft reconstitution. (A) RV.CBCFTR encodes the full length CFTR cDNA driven by the CMV/Beta-actin promoter. (B) RV.CFTR-PTM24 encodes the PTM-24 trans-splicing domain upstream to a partial cDNA of CFTR encoding exons 10-24 and driven by the CMV/Beta-actin promoter. (C) RV.CBLacZ encodes the full length LacZ cDNA driven by the CMV/Beta-actin promoter and serves as a control vector.

FIG. 9. PTM mediated delivery of CFTR improves airway epithelial reconstitution and differentiation in xenografts. Primary human bronchial airway epithelial cells were infected in vitro with three different retroviral vectors (RV.CBLacZ, RV.CBCFTR, RV.CFTRPTM-24) each with the same transcriptional elements (CMV enhancer/peta-actin promoter). (A) The level of integrated proviral genomes was quantified by Taq-Man PCR using primers specific for the CMV enhancer. Nearly equivalent levels of transduction were seen in these primary cultures. Primary airway epithelial cells (2×10⁶ cells) were then seeded into denuded rat tracheas at 3 days post-infection and subcutaneously implanted in nude athymic mice. Following 6 weeks of reconstitution, a fully differentiated airway epithelium is normally established in the xenograft rat tracheas. Xenografts were then harvested for generation of DNA and Taq-Man PCR quantification of viral genomes (Panel B) or histochemical staining for beta-galactosidase with X-gal (Panels C-E). Taq-Man PCR results demonstrated greater viral genome stability within epithelial DNA from xenografts infected with RV.CFTRPTM-24 as compared to RV.CBCFTR. There was still a slight decline in the abundance of RV.CFTRPTM-24 viral genomes as compared to RV.CBLacZ infected xenografts, suggesting that residual translation from the PTM vector produces a portion of the CFTR protein that may inhibit stem cell reconstitution. When the epithelium of reconstituted xenografts was evaluated, a striking difference in differentiation was seen between RV.CFTRPTM-24 (Panel D) and RV.CBCFTR (Panel E) infected xenografts. The height of the reconstituted epithelium (marked by a bracket), that is an indicator of stem cell proliferation, was significantly reduced in RV.CBCFTR as compared RV.CFTRPTM-24 infected xenografts. Furthermore, ciliated cells (ciliated apical surface is marked by arrows) were abundant in RV.CFTRPTM-24 infected xenografts and completely absent in RV.CBCFTR infected xenografts. Although the abundance of ciliated cells were similar between RV.CBLacZ (control vector) and RV.CFTRPTM-24 infected xenograft epithelium, the height of the epithelium was slightly reduced in RV.CFTRPTM-24 infected xenografts, supporting a reduced but not absent toxicity from the PTM-24 construct.

### 5. DETAILED DESCRIPTION OF THE INVENTION

The present invention relates to compositions comprising genetically engineered stem cells comprising a nucleic acid molecule capable of encoding pre-trans-splicing molecule (PTM) and the use of such cells for generating novel nucleic acid molecules designed to correct genetic defects. The PTMs expressed within the stem cell comprise (i) one or more target binding domains that are designed to specifically bind to a specific target pre-mRNA expressed within a stem cell and (ii) a 3′ splice region that includes a branch point and a 3′ splice acceptor site and/or a 5′ splice donor site. The 3′ splice region may further comprise a pyrimidine tract. In addition, the PTMs of the invention can be engineered to contain any nucleotide sequences such as those encoding a translatable protein product and one or more spacer regions that separate the RNA splice site from the target binding domain.

The methods of the invention encompass transferring a nucleic acid molecule capable of encoding a PTM into a stem cell under conditions in which the nucleic acid molecule is transcribed to express a PTM. The methods of the invention further comprise transplanting the genetically engineered stem cells into a subject host for correction of the genetic defect.

The present invention is based on the discovery that, in contrast to overexpression of CFTR in human airway stem cells which is toxic to the cell, expression of a CFTR corrective PTM is not associated with cytoxicity. Thus, the present invention provides a method based on trans-splicing for correcting genetic defects in stem cells where expression of mature stage proteins may be detrimental to maturation and proliferation of the stem cell.

### 5.1. Structure of the Pre-Trans-Splicing Molecules

The present invention provides compositions for use in generating novel chimeric nucleic acid molecules through targeted trans-splicing. The PTMs of the invention comprise (i) one or more target binding domains that targets binding of the PTM to a specific target pre-mRNA expressed within a differentiating stem cell and (ii) a 3′ splice region that includes a branch point and a 3′ splice acceptor site and/or 5′ splice donor site. The 3′ splice region may additionally contain a pyrimidine tract. The PTMs may also contain (a) one or more spacer regions that separate the RNA splice site from the target binding domain, (b) mini-intron sequences, (c) ISAR (intronic splicing activator and repressor) consensus binding sites, and/or (d) ribozyme sequences.

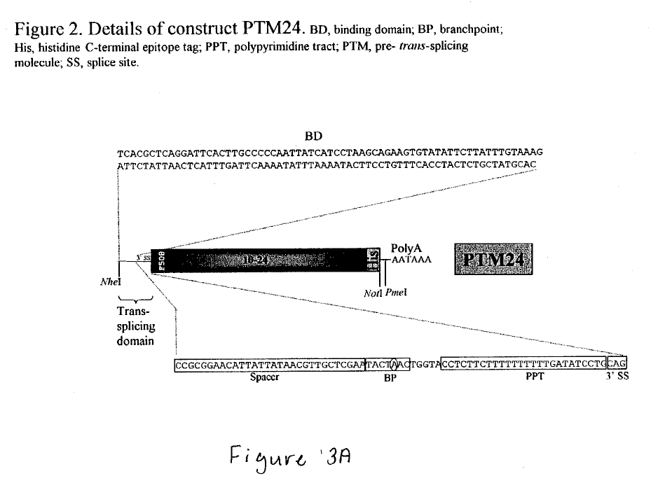

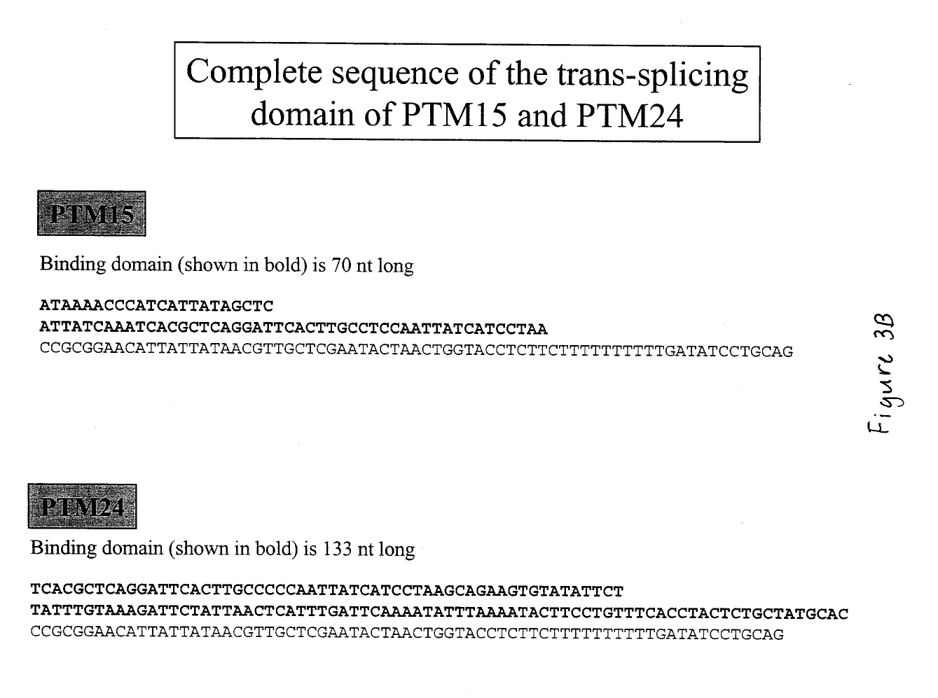

Additionally, the PTMs of the invention contain specific exon sequences designed to correct a specific genetic defect or add a new cellular function. The exon sequences used will depend on the genetic defect to be corrected. In a specific embodiment of the invention exon sequences designed to correct a cystic fibrosis transmembrane conductance regulator target pre-mRNA may be used such as those exon sequences included in the structure of the PTM 24 depicted in FIG. 3.

The PTMs of invention can also contain specific exon sequences to be used to correct genetic defects associated with a variety of different disorders such as cystic fibrosis, hemophilia, sickle cell anemia, Tay-Sachs disease, thalassemias, polycystic kidney disease and muscular dystrophy, to name a few.

A variety of different PTM molecules may be synthesized for use in the production of a novel chimeric RNA that complements a defective or inactive protein. The general design, construction and genetic engineering of such PTMs and demonstration of their ability to mediate successful trans-splicing reactions within the cell are described in detail in U.S. Pat. Nos. 6,083,702, 6,013,487 and 6,280,978 as well as patent Ser. Nos. 09/941,492, 09/756,095, 09/756,096 and 09/756,097 the disclosures of which are incorporated by reference in their entirety herein.

The target binding domain of the PTM endows the PTM with a binding affinity. As used herein, a target binding domain is defined as any molecule, i.e., nucleotide, protein, chemical compound, etc., that confers specificity of binding and anchors the specific target pre-mRNA closely in space to the PTM so that the spliceosome processing machinery of the nucleus can trans-splice a portion of the PTM to a portion of the specific target pre-mRNA. The target binding domain of the PTM may contain multiple binding domains which are complementary to and in anti-sense orientation to the targeted region of the selected pre-mRNA. The target binding domains may comprise up to several thousand nucleotides. In preferred embodiments of the invention the binding domains may comprise at least 10 to 30 and up to several hundred or more nucleotides. The specificity of the PTM may be increased significantly by increasing the length of the target binding domain. For example, the target binding domain may comprise several hundred nucleotides or more. In addition, although the target binding domain may be “linear” it is understood that the RNA may fold to form secondary structures that may stabilize the complex thereby increasing the efficiency of splicing. A second target binding region may be placed at the 3′ end of the molecule and can be incorporated into the PTM of the invention. Absolute complementarily, although preferred, is not required. A sequence “complementary” to a portion of an RNA, as referred to herein, means a sequence having sufficient complementarity to be able to hybridize with the target pre-RNA, forming a stable duplex. The ability to hybridize will depend on both the degree of complementarity and the length of the nucleic acid (See, for example, Sambrook et al., 1989, Molecular Cloning, A Laboratory Manual, 2d Ed., Cold Spring Harbor Laboratory Press, Cold Spring Harbor, N.Y.). Generally, the longer the hybridizing nucleic acid, the more base mismatches with an RNA it may contain and still form a stable duplex. One skilled in the art can ascertain a tolerable degree of mismatch or length of duplex by use of standard procedures to determine the stability of the hybridized complex.

Binding may also be achieved through other mechanisms, for example, through triple helix formation, aptamer interactions, antibody interactions or protein/nucleic acid interactions such as those in which the PTM is engineered to recognize a specific RNA binding protein, i.e., a protein bound to a specific target pre-mRNA. Alternatively, the PTMs of the invention may be designed to recognize secondary structures, such as for example, hairpin structures resulting from intramolecular base pairing between nucleotides within an RNA molecule.

In a specific embodiment of the invention, the target binding domain is complementary and in anti-sense orientation to sequences in close proximity to the region of the specific target pre-mRNA targeted for trans-splicing.

The PTM molecule also contains a 3′ splice region that includes a branch point sequence and a 3′ splice acceptor AG site and/or a 5′ splice donor site. The 3′ splice region may further comprise a pyrimidine tract. Consensus sequences for the 5′ splice donor site and the 3′ splice region used in RNA splicing are well known in the art (See, Moore, et al., 1993, The RNA World, Cold Spring Harbor Laboratory Press, p. 303-358). In addition, modified consensus sequences that maintain the ability to function as 5′ donor splice sites and 3′ splice regions may be used in the practice of the invention. Briefly, the 5′ splice site consensus sequence is AG/GURAGU (where A=adenosine, U=uracil, G=guanine, C=cytosine, R=purine and/=the splice site). The 3′ splice site consists of three separate sequence elements: the branch point or branch site, a polypyrimidine tract and the 3′ consensus sequence (YAG). The branch point consensus sequence in mammals is YNYURAC (Y=pyrimidine; N=any nucleotide). The underlined A is the site of branch formation. A polypyrimidine tract is located between the branch point and the splice site acceptor and is important for different branch point utilization and 3′ splice site recognition. Recently, pre-messenger RNA introns beginning with the dinucleotide AU and ending with the dinucleotide AC have been identified and referred to as U12 introns. U12 intron sequences as well as any sequences that function as splice acceptor/donor sequences may also be used to generate the PTMs of the invention.

A spacer region to separate the RNA splice site from the target binding domain may also be included in the PTM. The spacer region may be designed to include features such as stop codons which would block translation of an unspliced PTM and/or sequences that enhance trans-splicing to the target pre-mRNA.

In a preferred embodiment of the invention, a “safety” is also incorporated into the spacer, binding domain, or elsewhere in the PTM to prevent non-specific trans-splicing. This is a region of the PTM that covers elements of the 3′ and/or 5′ splice site of the PTM by relatively weak complementarity, preventing non-specific trans-splicing. The PTM is designed in such a way that upon hybridization of the binding/targeting portion(s) of the PTM, the 3′ and/or 5′splice site is uncovered and becomes fully active.

The “safety” consists of one or more complementary stretches of cis-sequence (or could be a second, separate, strand of nucleic acid) which weakly binds to one or both sides of the PTM branch point, pyrimidine tract, 3′ splice site and/or 5′ splice site (splicing elements), or could bind to parts of the splicing elements themselves. This “safety” binding prevents the splicing elements from being active (i.e., block U2 snRNP or other splicing factors from attaching to the PTM splice site recognition elements). The binding of the “safety” may be disrupted by the binding of the target binding region of the PTM to the target pre-mRNA, thus exposing and activating the PTM splicing elements (making them available to trans-splice into the target pre-mRNA).

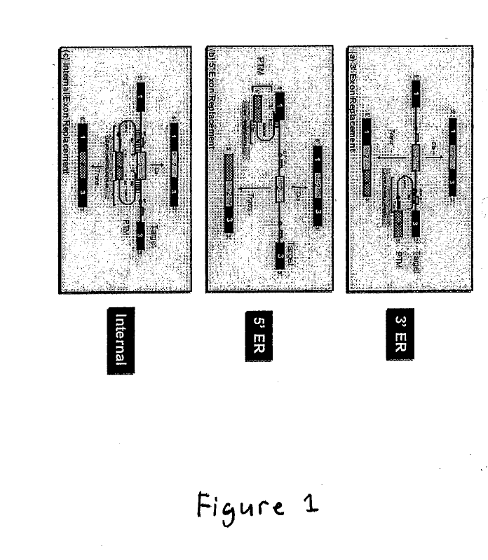

The PTM's of the invention may be engineered to contain a single specific exon sequence, multiple specific exon sequences, or alternatively a complete set of specific exon sequences. The number and identity of the specific sequences to be used in the PTMs will depend on the targeted mutation to be corrected, and the type of trans-splicing reaction, i.e., 5′ exon replacement, 3′ exon replacement or internal exon replacement that will occur (see FIG. 1). In addition, to limit the size of the PTM, the molecule may include deletions in non-essential regions of the specific gene.

The present invention further provides PTM molecules wherein the coding region of the PTM is engineered to contain mini-introns. The insertion of mini-introns into the coding sequence of the PTM is designed to increase definition of the exon and enhance recognition of the PTM donor site. Mini-intron sequences to be inserted into the coding regions of the PTM include small naturally occurring introns or, alternatively, any intron sequences, including synthetic mini-introns, which include 5′ consensus donor sites and 3′ consensus sequences which include a branch point, a 3′splice site and in some instances a pyrimidine tract.

The mini-introns sequences are preferably between about 60-100 nucleotides in length, however, mini-intron sequences of increased lengths may also be used. In a preferred embodiment of the invention, the mini-intron comprises the 5′ and 3′ end of an endogenous intron. In preferred embodiments of the invention, the 5′ intron fragment is about 20 nucleotides in length and the 3′ end is about 40 nucleotides in length.

In a specific embodiment of the invention, an intron of 528 nucleotides comprising the following sequences may be utilized. Sequence of the intron construct is as follows:

5′ fragment sequence:

Gtagttcttttgttcttcactattaagaacttaatttggtgtccatgtctctttttttttctagtttgtagtgctggaag gtatttttggagaaattcttacatgagcattaggagaatgtatgggtgtagtgtcttgtataatagaaattgttccactgataatttactct agttttttatttcctcatattattttcagtggctttttcttccacatctttatattttgcaccacattcaacactgtagcggccgc.

3′ fragment sequence:

Ccaactatctgaatcatgtgccccttctctgtgaacctctatcataatacttgtcacactgtattgtaattgtctct tttactttcccttgtatcttttgtgcatagcagagtacctgaaacaggaagtattttaaatattttgaatcaaatgagttaatagaatcttta caaataagaatatacacttctgcttaggatgataattggaggcaagtgaatcctgagogtgatttgataatgacctaataatgatggg ttttatttccag

In yet another specific embodiment of the invention, consensus ISAR sequences are included in the PTMs of the invention (Jones et al., NAR 29:3557-3565). Proteins bind to the ISAR splicing activator and repressor consensus sequence which includes a uridine-rich region that is required for 5′ splice site recognition by U1 SnRNP. The 18 nucleotide ISAR consensus sequence comprises the following sequence: GGGCUGAUUUUUCCAUGU. When inserted into the PTMs of the invention, the ISAR consensus sequences are inserted into the structure of the PTM in close proximity to the ₅′ donor site of intron sequences. In an embodiment of the invention the ISAR sequences are inserted within 100 nucleotides from the 5′ donor site. In a preferred embodiment of the invention the ISAR sequences are inserted within 50 nucleotides from the 5′ donor site. In a more preferred embodiment of the invention the ISAR sequences are inserted within 20 nucleotides of the 5′ donor site.

The compositions of the invention further comprise PTMs that have been engineered to include cis-acting ribozyme sequences. The inclusion of such sequences is designed to reduce PTM translation in the absence of trans-splicing. The ribozyme sequences that may be inserted into the PTMs include any sequences that are capable of mediating a cis-acting (self-cleaving) RNA splicing reaction. Such ribozymes include but are not limited to hammerhead, hairpin and hepatitis delta virus ribozymes (see, Chow et al. 1994, J Biol Chem 269:25856-64).

In an embodiment of the invention, splicing enhancers such as, for example, sequences referred to as exonic splicing enhancers may also be included in the structure of the PTMs. Transacting splicing factors, namely the serine/arginine-rich (SR) proteins, have been shown to interact with such exonic splicing enhancers and modulate splicing (See, Tacke et al., 1999, Curr. Opin. Cell Biol. 11:358-362; Tian et al., 2001, J. Biological Chemistry 276:33833-33839; Fu, 1995, RNA 1:663-680). Nuclear localization signals may also be included in the PTM molecule (Dingwell and Laskey, 1986, Ann. Rev. Cell Biol. 2:367-390; Dingwell and Laskey, 1991, Trends in Biochem. Sci. 16:478-481). Such nuclear localization signals can be used to enhance the transport of synthetic PTMs into the nucleus where trans-splicing occurs.

Additional features can be added to the PTM molecule either after, or before, the nucleotide sequence encoding a translatable protein, such as polyadenylation signals or 5′ splice sequences to enhance splicing, additional binding regions, “safety”-self complementary regions, additional splice sites, or protective groups to modulate the stability of the molecule and prevent degradation.

PTMs may also be generated that require a double-trans-splicing reaction for generation of a chimeric trans-spliced product. Such PTMs could be used to replace an internal exon which could be used for specific gene repair. PTMs designed to promote two trans-splicing reactions are engineered as described above, however, they contain both 5′ donor sites and 3′ splice acceptor sites. In addition, the PTMs may comprise two or more binding domains and splicer regions. The splicer regions may be place between the multiple binding domains and splice sites or alternatively between the multiple binding domains.

### 5.2. Synthesis of the Trans-Splicing Molecules

For production of a nucleic acid molecule encoding a PTM, cloning techniques known in the art may be used for cloning of the nucleic acid molecule into an expression vector. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

The DNA encoding the PTM of interest may be recombinantly engineered into a variety of host vector systems that also provide for replication of the DNA in large scale and contain the necessary elements for directing the transcription of the PTM. The use of such a construct to transfect target stem cells will result in the transcription of sufficient amounts of PTMs that will form complementary base pairs with the endogenously expressed specific target pre-mRNA and thereby facilitate a trans-splicing reaction between the complexed nucleic acid molecules. For example, a vector can be introduced in vivo such that it is taken up by a cell and directs the transcription of the PTM molecule. Such a vector can remain episomal or become chromosomally integrated, as long as it can be transcribed to produce the desired RNA, i.e., PTM. Such vectors can be constructed by recombinant DNA technology methods standard in the art.

Vectors encoding the PTM of interest can be plasmid, viral, or others known in the art, used for replication and expression in mammalian cells. Such vectors include any eukaryotic expression vectors, including but not limited to viral expression vectors such as those derived from the class of retroviruses, adenoviruses or adeno-associated viruses. Expression of the sequence encoding the PTM can be regulated by any promoter/enhancer sequences known in the art to act in mammalian, preferably human cells. Such promoters/enhancers can be inducible or constitutive. Such promoters include but are not limited to: the SV40 early promoter region (Benoist, C. and Chambon, P. 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the viral CMV promoter, the human β-chorionic gonadotropin-6 promoter (Hollenberg et al., 1994, Mol. Cell. Endocrinology 106:111-119), etc. In a preferred embodiment of the invention, promoter/enhancer sequences may be used to promote the of PTMs in the cell type to which the stem cell is expected to differentiate into, i.e., lung tissue, blood cells, etc.

### 5.3. Uses and Administration of Trans-Splicing Molecules

The compositions and methods of the present invention can be utilized to correct specific genetic defects. Specifically, targeted trans-splicing, including double-trans-splicing reactions, 3′ exon replacement and/or 5′ exon replacement can be used to repair or correct specific transcripts that are either truncated or contain point mutations leading to a mutant phenotype. The PTMs of the invention are designed to cleave a targeted transcript upstream or downstream of a specific mutation or upstream of a premature 3′ stop codon and correct the mutant transcript via a trans-splicing reaction which replaces the portion of the transcript containing the mutation with a functional sequence.

In particular, the present invention relates to the transfer of a nucleic acid molecule capable of expressing a PTM into a targeted stem cell. Upon differentiation, the stem cell will begin to express the target pre-mRNA thereby providing substrate for a PTM mediated trans-splicing reaction. As used herein the term stem cell refers to any pluripotent or multipotent progenitor cell that has retained the ability to replicate and differentiate into different cell lineages. Such cells include, but are not limited to, ES cells, hematopoeitic stem cells, human amniotic epithelial cells, mesenchymal stem cells and hepatic oval cells to name a few. Methods for enriching for populations of stem cells derived from a subject are well known to those of skill in the art. Such methods include but are not limited to those that rely on the use of antibodies that recognize stem cell surface markers.

Stem cells may be obtained from a variety of different donor sources. In a preferred embodiment, autologous stem cells are obtained from the subject who is to receive the engineered stem cells. This approach is especially advantageous since the immunological rejection of foreign tissue and/or a graft versus host response is avoided. In yet another preferred embodiment of the invention, allogenic stem cells may be obtained from donors who are genetically related to the recipient and share the same transplantation antigens on the surface of their stem cells. Alternatively, if a related donor is unavailable, stem cells from antigenically matched (identified through a national registry) donors may be used.

Stem cells can be obtained from the donor by standard techniques known in the art. For example, bone marrow stem cells can be removed from the donor by placing a hollow needle into the marrow space and withdrawing a quantity of marrow cells by aspiration. Alternatively, peripheral stem cells can be obtained from a donor, for example, by standard phlebotomy or apheresis techniques. In yet another embodiment of the invention, stem cells may be derived from tissue samples known to contain progenitor stem cells. This may be readily accomplished using techniques known to those skilled in the art. For example, the tissue can be disaggregated mechanically and/or treated with digestive enzymes and/or chelating agents that weaken the connections between neighboring cells, making it possible to disperse the tissue suspension of individual cells. Enzymatic dissociation can be carried out by mincing the tissue and treating the minced tissue with any of a number of digestive enzymes. Such enzymes include, but are not limited to, trypsin, chymotrpsin, collagenase, elastase and/or hylauronidase. A review of tissue disaggregation technique is provided in, e.g., Freshney, Culture of Animal Cells, A Manual of Basic Technique, 2d Ed., A. R. Liss, Inc., New York, 1987, Ch. 9, pp.107-126.

Stem cell populations maybe enriched for by selecting for cells that express stem cell surface antigens, in combination with purification techniques such as immuno-magnetic bead purification, affinity chromatography and fluorescence activated cell sorting. The expressed stem cell surface antigen screened for will depend on the type of stem cell to be utilized. Such cell surface antigens are known in the art.

Various delivery systems are known and can be used to transfer nucleic acid molecules encoding PTMs into stem cells, e.g. encapsulation in liposomes, microparticles, microcapsules, recombinant cells capable of expressing the composition, receptor-mediated endocytosis (see, e.g., Wu and Wu, 1987, J. Biol. Chem. 262:4429-4432), construction of a nucleic acid as part of a retroviral adenoviral, adeno-associated viral or other vector, injection of DNA, electroporation, calcium phosphate mediated transfection, etc.

The compositions and methods can be used to provide sequences encoding a functional biologically active molecule to cells of an individual with an inherited genetic disorder where expression of the missing or mutant specific gene product produces a normal phenotype.

In a preferred embodiment, nucleic acids comprising a sequence encoding a PTM are administered to promote PTM function, by way of gene delivery and expression into a host stem cell or progenitor cell. In this embodiment of the invention, the nucleic acid mediates an effect by promoting PTM production. Any of the methods for gene delivery into a host cell available in the art can be used according to the present invention. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215. Exemplary methods are described below.

In a specific embodiment, a viral vector that contains the PTM can be used. For example, a retroviral vector can be utilized that has been modified to delete retroviral sequences that are not necessary for packaging of the viral genome and integration into host cell DNA (see Miller et al., 1993, Meth. Enzymol. 217:581-599). Alternatively, adenoviral or adeno-associated viral vectors can be used for gene delivery to cells or tissues. (See, Kozarsky and Wilson, 1993, Current Opinion in Genetics and Development 3:499-503 for a review of adenovirus-based gene delivery).

In a preferred embodiment of the invention an adeno-associated viral vector may be used to deliver nucleic acid molecules capable of encoding the PTM. The vector is designed so that, depending on the level of expression desired, the promoter and/or enhancer element of choice may be inserted into the vector.

In a specific embodiment of the invention, gene delivery into a stem cell involves transferring a nucleic acid molecule capable of encoding a PTM to stem cells or progenitor cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. The resulting recombinant stem cells can be delivered to a host by various methods known in the art. In a preferred embodiment, the cell used for gene delivery is autologous to the host.

In a specific embodiment of the invention, stem cells may be derived from a subject having a genetic disorder and transfected with a nucleic acid molecule capable of encoding a PTM designed to correct the specific genetic disorder. Alternatively, stem cells may be infected with recombinant viruses engineered to encode a PTM. Cells may be further selected, using routine methods known to those of skill in the art, for integration of the nucleic acid molecule into the genome thereby providing a stable stem cell culture expressing the PTM of interest. Such cells are then transplanted into the subject thereby providing a source of corrected protein.

In addition, stem cells may be attached in vitro to a natural or synthetic matrix that provides support for the transplanted cells prior to transplantation. The type of matrix that may be used in the practice of the invention is virtually limitlessness. The matrix will have all the features commonly associated with being biocompatible, in that it is in a form that does not produce an adverse, or allergic reaction when administered to the recipient host. Growth factors capable of stimulating the growth and regeneration of the desired tissue may also be incorporated into the matrices. Such matrices may be formed from both natural or synthetic materials and may be designed to allow for sustained release of growth factors over prolonged periods of time. Thus, appropriate matrices will both provide growth factors and also act as an in situ scaffolding in which the transplanted cells differentiate and proliferate to form new tissue. In preferred embodiments, it is contemplated that a biodegradable matrix that is capable of being reabsorbed into the body will likely be most useful.

The present invention also provides for pharmaceutical compositions comprising stem cells expressing an effective amount of a PTM and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Examples of suitable pharmaceutical carriers are described in “Remington's Pharmaceutical sciences” by E. W. Martin.

In specific embodiments, pharmaceutical compositions are administered in diseases or disorders involving an absence or decreased (relative to normal or desired) level of an endogenous protein or function, for example, in hosts where the specific protein is lacking, genetically defective, biologically inactive or underactive, or under expressed. Such genetic disorders include but are not limited to cystic fibrosis, hemophilia, sickle cell anemia, Tay-Sachs disease, thalassemias, polycystic kidney disease, and muscular dystrophy. The activity of the specific protein encoded for by the chimeric mRNA resulting from the PTM mediated trans-splicing reaction can be readily detected, e.g., by obtaining a host tissue sample (e.g., from biopsy tissue) and assaying it in vitro for mRNA or protein levels, structure and/or activity of the expressed chimeric mRNA. Many methods standard in the art can be thus employed, including but not limited to immunoassays to detect and/or visualize the protein encoded for by the chimeric mRNA (e.g., Western blot, immunoprecipitation followed by sodium dodecyl sulfate polyacrylamide gel electrophoresis, immunocytochemistry, etc.) and/or hybridization assays to detect formation of chimeric mRNA expression by detecting and/or visualizing the presence of chimeric mRNA (e.g., Northern assays, dot blots, in situ hybridization, and reverse-transcription PCR, etc.), etc.

In a specific embodiment, it may be desirable to administer the pharmaceutical compositions of the invention locally to the area in need of treatment. This may be achieved by, for example, and not by way of limitation, topical application, e.g., in conjunction with a wound dressing after surgery, by injection, or by means of an implant, said implant being of a porous, non-porous, or gelatinous material, including membranes, such as sialastic membranes, or fibers. Other control release drug delivery systems, such as nanoparticles, matrices such as controlled-release polymers, hydrogels.

The genetically modified stem cells will be administered in amounts which are effective to produce the desired effect in the host. Effective dosages of the stem cells can be determined through procedures well known to those in the art which address such parameters as biological half-life, bioavailability and toxicity. The amount of the composition of the invention which will be effective will depend on the type and severity of the genetic disorder being treated, and can be determined by standard clinical techniques. Such techniques include analysis of tissue samples to determine levels of protein expression. In addition, in vitro assays may optionally be employed to help identify optimal dosage ranges.

The present invention also provides a pharmaceutical pack or kit comprising one or more containers filled with one or more of the ingredients of the pharmaceutical compositions of the invention optionally associated with such container(s) can be a notice in the form prescribed by a governmental agency regulating the manufacture, use or sale of pharmaceuticals or biological products, which notice reflects approval by the agency of manufacture, use or sale for human administration.

### 6. EXAMPLE: EXPRESSION OF FULL-LENGTH CFTR 1N STEM CELLS

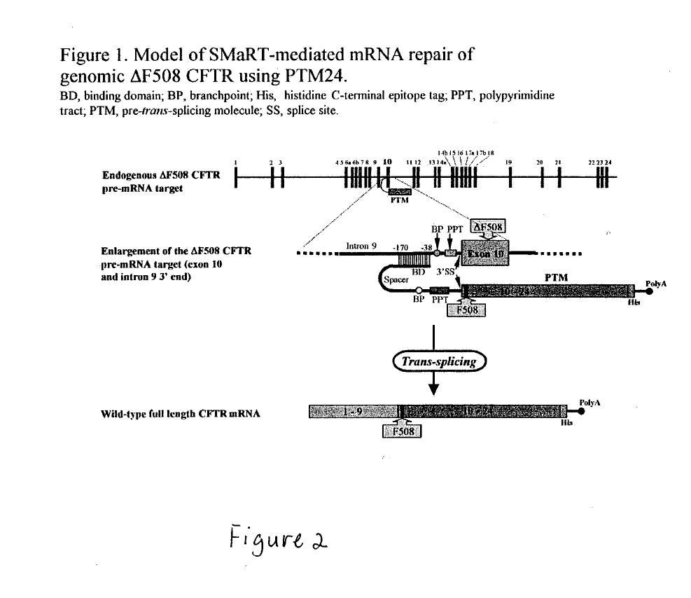

FIG. 2 represents the overall protocol used to assess CFTR stem cell reconstitution in airway stem cells. Primary airway epithelial cells were transduced with either LacZ or CFTR expressing retroviruses. Transgene expression was assessed by Southern blot analysis or X-gal staining (typically 10-20% of the cells will stain). Primary cells were then seeded into human bronchial xenografts and allowed to reconstitute for 5-6 weeks. Xenograft airway epithelium was then assessed for CFTR or LacZ transgene expression.

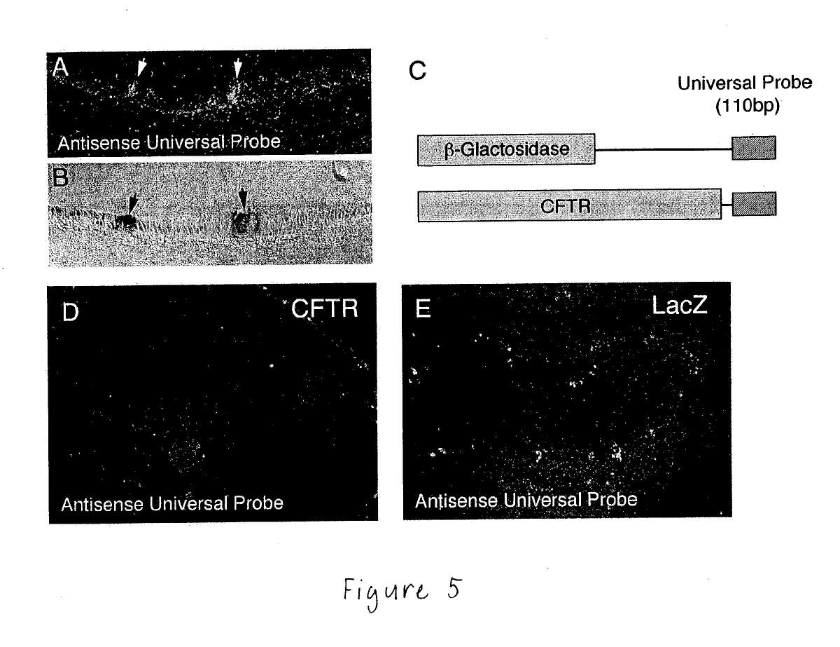

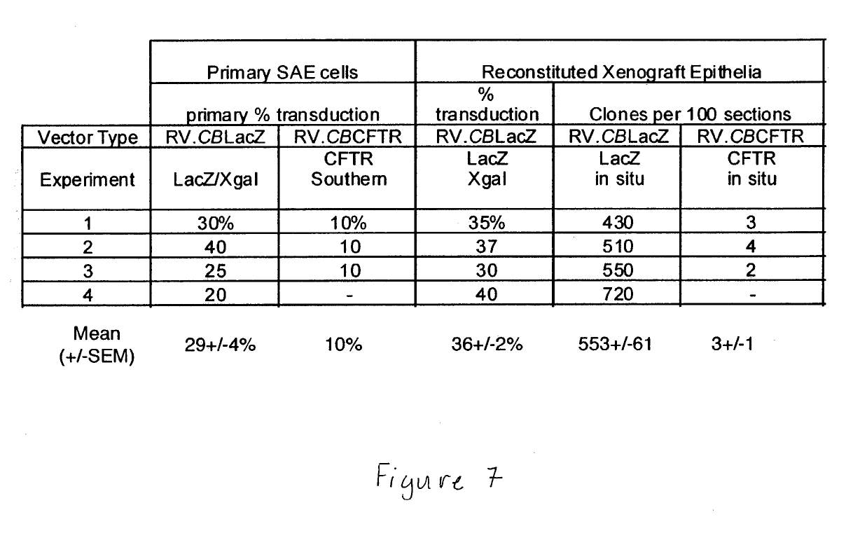

To directly compare transgene expression from xenografts infected with CFTR or LacZ expressing retroviruses, an in situ expression assay was developed using a RNA probe complementary to the 3′-untranslated region of both vector transgenes. This is referred to as the “Universal Probe” and its position is indicated in FIG. 5C. To test the sensitivity of this probe, serial sections from RV.CBLacZ reconstituted xenografts were used to detect expression of the transgene by in situ hybridization (FIG. 5A) or X-gal histochemical staining (FIG. 5B). Results demonstrate a complete concordance in clone location and sensitivity, indicating that this approach could be used successfully for comparing CFTR to LacZ gene expression. In FIGS. 5D and 5E, xenografts reconstituted with either RV.CBCFTR (D) or RV.CBLacZ (E) retroviruses were sectioned and probed with antisense Universal Probe to detect transgene expression. As indicated, many more clones exist in the surface airway epithelium in RV.CBLacZ infected xenografts as compared to RV.CBCFTR. White grains indicated expression of mRNA target. Quantification of the results is presented in FIG. 7.

A summary of LacZ and CFTR expression in SAE cells of xenografts using conventional retroviral vectors is presented in FIG. 7. The figure represents transgene expression in primary airway epithelia following infection in vitro and the level of sustained expression following reconstitution of airway epithelia in xenografts. The two vectors used for infection were RV.CBCFTR and RV.CBLacZ. X-gal staining of primary epithelial cells and reconstituted xenografts was used to determine the level of expression prior to seeding in xenografts and the level of clonal expansion following reconstitution of xenografts. Additionally, comparison to CFTR infected xenografts, in situ hybridization was also used to assess expression in xenografts infected with RV.CBLacZ. In contrast, Southern blot analysis was used to determine the level of infection in primary airway epithelial cells with the RV.CBCFTR vector and in situ hybridization was used to assess expression from this vector following reconstitution in xenografts.

A summary of levels of expression is given below the table. The results demonstrate a significantly lower level of full length CFTR reconstitution in xenografts as compared to LacZ. Assessing comparative clonal expansion suggest that a greater than 100-fold selective disadvantage is encountered for stem cell reconstitution of CFTR as compared to LacZ.

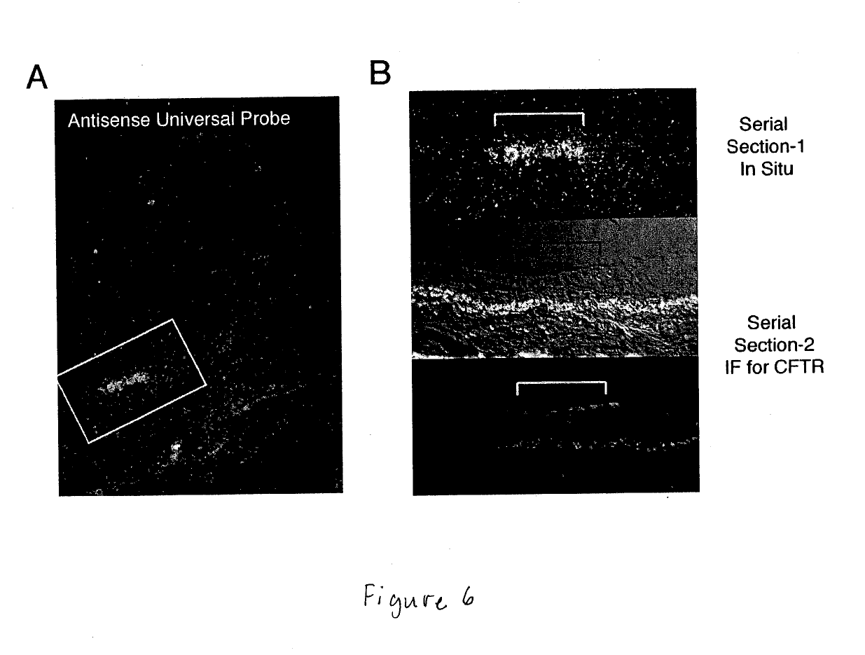

Despite the low abundance of CFTR expression from a retroviral vector (RV.CBCFTR) expressing the full-length CFTR cDNA, expression of both CFTR transgene derived mRNA and CFTR protein could be detected in infrequent non-ciliated cell clones. FIG. 6 demonstrates in situ hybridization using the antisense universal probe to the 3′ untranslated region of the retrovirally derived CFTR transgene. One clone expressing CFTR mRNA is boxed in FIG. 6A. A serial section from that shown in FIG. 6A was stained for CFTR protein. Both the serial in situ staining and CFTR protein staining are shown in FIG. 6B (top and bottom panel). The middle panel of 6B is a Nomarski photomicrograph of the same field in the bottom panel. This demonstrates that cells that express CFTR have an undifferentiated non-ciliated phenotype and implies that high level CFTR expression may be deleterious to stem cells and their ability to differentiate. Hence, overexpression of CFTR is airway stem cells appears to be toxic.

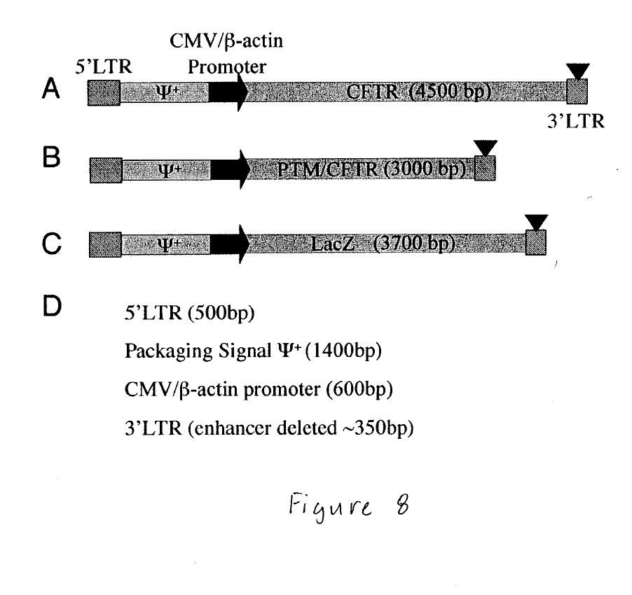

Retroviral vectors were developed to test delivery of a CFTR PTM. Three retroviral vectors were compared for their ability to reconstitute transgene expression in airway stem cells by xenograft reconstitution. FIG. 8A depicts RV.CBCFTR which encodes the full length CFTR cDNA driven by the CMV/Beta-actin promoter. RV.CFTR-PTM24 encodes the PTM-24, depicted in FIG. 8B, trans-splicing domain upstream to a partial cDNA of CFTR encoding exons 10-24 and driven by the CMV/Beta-actin promoter. RV.CBLacZ as depicted in FIG. 8C, encodes the full length LacZ cDNA driven by the CMV/Beta-actin promoter and serves as a control vector.

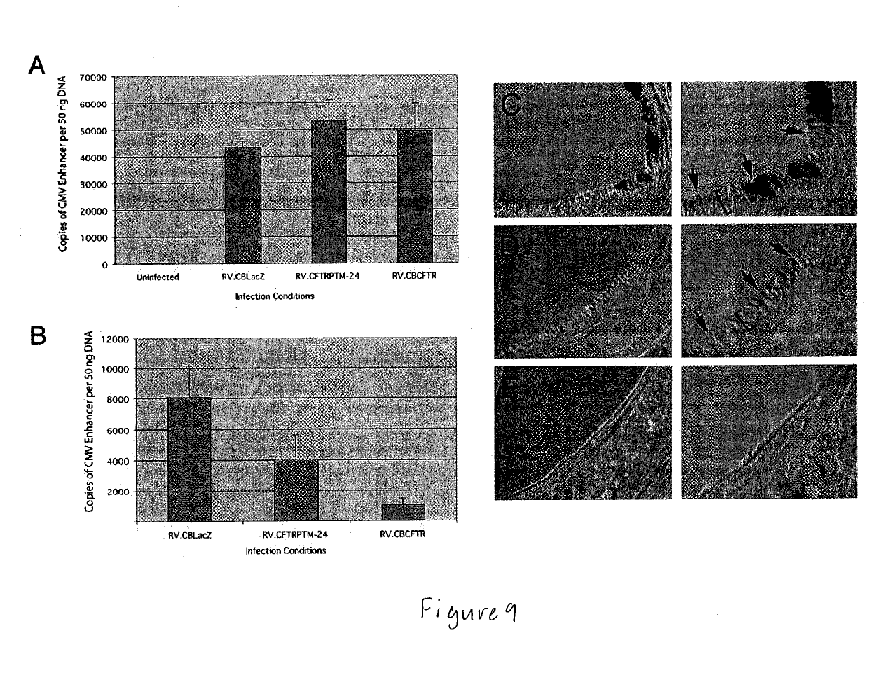

As depicted in FIG. 9, PTM mediated delivery of CFTR improves airway epithelial reconstitution and differentiation in xenografts. Primary human bronchial airway epithelial cells were infected in vitro with three different retroviral vectors (RV.CBLacZ, RV.CBCFTR, RV.CFTRPTM-24) each with the same transcriptional elements (CMV enhancer/βeta-actin promoter). FIG. 9A depicts the level of integrated proviral genomes was quantified by Taq-Man PCR using primers specific for the CMV enhancer. Nearly equivalent levels of transduction were seen in these primary cultures. Primary airway epithelial cells (2×10⁶ cells) were then seeded into denuded rat tracheas at 3 days post-infection and subcutaneously implanted in nude athymic mice. Following 6 weeks of reconstitution, a fully differentiated airway epithelium is normally established in the xenograft rat tracheas. Xenografts were then harvested for generation of DNA and Taq-Man PCR quantification of viral genomes (FIG. 9B) or histochemical staining for beta-galactosidase with X-gal (FIGS. 9C-E). Taq-Man PCR results demonstrated greater viral genome stability within epithelial DNA from xenografts infected with RV.CFTRPTM-24 as compared to RV.CBCFTR. There was still a slight decline in the abundance of RV.CFTRPTM-24 viral genomes as compared to RV.CBLacZ infected xenografts, suggesting that residual translation from the PTM vector produces a portion of the CFTR protein that may inhibit stem cell reconstitution. When the epithelium of reconstituted xenografts was evaluated, a striking difference in differentiation was seen between RV.CFTRPTM-24 (FIG. 9D) and RV.CBCFTR (FIG. 9E) infected xenografts. The height of the reconstituted epithelium (marked by a bracket), that is an indicator of stem cell proliferation, was significantly reduced in RV.CBCFTR as compared RV.CFTRPTM-24 infected xenografts. Furthermore, ciliated cells (ciliated apical surface is marked by arrows) were abundant in RV.CFTRPTM-24 infected xenografts and completely absent in RV.CBCFTR infected xenografts. Although the abundance of ciliated cells were similar between RV.CBLacZ (control vector) and RV.CFTRPTM-24 infected xenograft epithelium, the height of the epithelium was slightly reduced in RV.CFTRPTM-24 infected xenografts, supporting a reduced but not absent toxicity from the PTM-24 construct. These findings are consistent with the Taq-Man PCR data demonstrating a slightly reduced stability of vector genomes when comparing RV.CFTRPTM-24 to RV.CBLacZ infection. These data clearly demonstrate that SMART delivery of CFTR can reduce the toxicity accompanying high level ectopic CFTR expression in airway stem cells. This toxicity appears to be associated with a reduced capacity of airway stem cells to both differentiation and proliferate.

### 7. EXAMPLE: TRANS-SPLICING MEDIATED REPAIR OF THE F508 CFTR MUTATION

Conditional repair and expression of CFTR by trans-splicing in ΔF508/ΔF508 pulmonary stem cells. Primary human surface airway epithelial (SAE) cells from CF patients are transduced with retroviruses that encode either a CFTR correcting PTM or LacZ. The transduced cells are tested for their potential to expand and survive in an in vivo airway reconstitution xenograft models. The percentage of transduced primary cells for each delivered gene (CFTR-PTM or LacZ) is compared to the number of transgene positive clones present in reconstituted xenograft bronchial epithelia. Following reconstitution of adult human bronchial xenografts with lacZ or CFTR transduced primary cells, xenografts are evaluated at 5 weeks post-transplantation for (i) CFTR chloride channel activity, (ii) LacZ transgene expression using histochemical staining, (iii) CFTR transgene expression using immunohistochemistry, and (iv) transgene derived LacZ and PTM CFTR mRNA using in situ hybridization.

As demonstrated in Example 6, ectopic CFTR expression in airway stem cells confers a selective disadvantage to reconstitution and persistence of the transgene in xenograft airway epithelium. One advantage associated with PTM mediated gene repair is that expression of normal CFTR is suppressed until differentiating stem cells begin to naturally express mutant CFTR, thus eliminating this selective disadvantage in corrected stem cells. Trans-splicing offers a solution to this potentially serious flaw in gene therapies designed to target stem cells in CF patients, and may also have application in preventing deleterious gene expression in stem cell therapy of other genetic diseases.

CF primary airway cells with a defined Δ F508 genotype are infected with concentrated retroviral stocks. Typical titers of retroviral stocks to be employed are 1×10⁸ cfu/ml. LacZ retroviral stocks tested at this titer can transduce 100% of primary cells in culture leading to nearly complete reconstitution of xenografts, determined using LacZ. Procedures for generation of VSV-G pseudotyped retroviral vectors are outlined below.

Two retroviral vectors may be used to transfer the CF-PTM and LacZ transgenes into primary human airway stem cell in vitro. Each of these vectors contain the CMV enhancer, Peta-actin promoter, and SV40 poly A sequences. All recombinant retroviral producer cell lines are tested for the presence of helper virus based on a lacZ mobilization assay. Primary cells are infected with retroviral producer supernatants in the presence of 2 Mg/ml polybrene. These cells are used to reconstitute denuded rat tracheas as described below.

Retroviral vectors for bronchial xenograft transduction and reconstitution are presented in FIG. 8. As indicated, each vector harbors identical regulatory elements. A deletion within the 3′-LTR inactivates the LTR as a promoter following integration. This construct design is used for cloning CF-PTMs as described.

For vector production, pBabePuroEBV-based retrovirus shuttle plasmids modified to contain a multiple cloning site are used. The plasmid contains an EBV origin of replication for in vivo replication, the puromycin resistance gene for selection, and a modified cloning site for expression of transgene off the viral LTR. CF-PTM 5, 15, and 16 are each cloned into this virus and tested. Optimized CF-PTMs may also be tested.

Amphotropic and ecotropic cells lines, the Phoenix™-A or -E, respectively, were utilized for production of retroviral vectors. These are second generation, 293 based BOSC and BING lines that provide for high titer retroviral production. Cell lines that may be used include, for example, GP2-293 (Clontech:Palo Alto, Calif.). Briefly, cells are transfected with purified plasmid DNA containing the construct of interest. BES buffered saline is used for transfection of 293 cells, with a resultant transfection efficiency of 70 to 80%. Supernatants are harvested after cells reach confluence. Supernatants are tested for transgene following infection of NIH3T3 cells or HeLa cells. Producer lines are expanded after 3 cycles of puromycin selection, which is added 48 to 72 hr after transfection. Selected cells are not subcloned unless individual titers are extremely low. These methods generate working titers of 105 to 106 cfu/ml. The cell line TA7 is also available for retroviral production. This cell line can be used to produce retrovirus titers which average 108 cfu/ml. Supernatants collected from multiple plates are concentrated if necessary.

For higher titer preparations, retrovirus-containing culture medium is clarified from cell debris by filtration and centrifugation, with the pellet resuspended in 0.5 ml of Hank's balanced salt solution. Titering is done on NIH3T3 cells. At the time of use, samples are re-titered on the appropriate cells line (NIH3T3 cells for amphotropic, VSV-G; HeLa for xenotropic, etc.). For purification, concentrated retrovirus suspensions are loaded onto 25-40% discontinuous sucrose gradients. Gradients are centrifuged in a SW-41 rotor at 40,000 rpm for 1.5 h at 4° C. Virus is collected from the interface between two layers of sucrose and the buffer is exchanged to lactose storage buffer by diafiltration through Filtron 100K concentrators, titrated and stored at −80° C. This method of concentration can increase viral titers three orders of magnitude (10⁷-10⁹ cfu/ml) depending on the amount of viral supernatant used for concentration.

Initial retrovirus is constructed to deliver CFTR targeted PTM 15 and PTM 24 (FIG. 3B). Primary CF airway epithelial cells are infected with concentrated retroviral stock and seeded into denuded rat tracheal xenografts. Epithelial transduction as assessed in primary cells following infection using Southern blot analysis for integrated viral transgenes. Five weeks following seeding of primary cells into xenografts, functional measurements for CFTR induced chloride permeability changes in response to cAMP agonists using transepithelial potential differences (PDs) are performed as described below. cAMP induced changes in transepithelial PD is compared between CF-PTM and LacZ transduced xenografts.

Human bronchial xenografts are generated from retrovirally transduced primary surface airway epithelial (SAE) cells derived from lung transplant tissue. Human bronchial SAE cells are harvested by treatment with 0.1% protease-14 in MEM for 36 hrs followed by agitation. Cells are then washed twice with 10% FCS/MEM and plated in hormonally defined F12 7×medium. Purified populations of SAE cells are retrovirally infected with amphotropic producer supernatants for 2 hrs on three consecutive days. Cells are harvested by treatment with trypsin-EDTA and seeded at a density of 1×106 cells into donor Fisher rat tracheas which are denuded of all viable epithelium by freeze thawing three times and rinsing in MEM. Previous reports using human surface bronchial epithelial cell population to seed denuded rat tracheas have demonstrated the utility of this model in generating a fully differentiated epithelium as early as 4 weeks post-transplantation, which is functionally equivalent to that of a native human bronchus with respect to electrophysiology and expression of the cell specific markers cytokeratin-14, cytokeratin-18, and CFTR.

Following infection of primary cells with LacZ and CF-PTM retroviral vectors, the percentage of transduced SAE cells is evaluated by Southern blot analysis for integrated viral genomes. Additionally, the percentages of cells expressing CFTR and LacZ transgenes is evaluated by histochemical staining for β-galactosidase and immunofluorescence for CFTR expression. Protocols for these assays are as previously described. Following reconstitution of bronchial xenografts with primary SAE cells, xenograft tissues are harvested at 5 weeks post-transplantation and expression of both LacZ and CFTR transgenes determined by in situ hybridization for mRNA using probes within both CFTR and LacZ 3′-untraslated regions, histochemical staining using X-gal for LacZ, and immunofluorescence for CFTR using alpha-1468 antibody which detects the C-terminus of the CFTR protein.

Transepithelial potential difference (PD) is used to assess the level of correction in cAMP-induced C, 1 permeability at 5 weeks post-transplantation. Measurements of transepithelial PDs are performed as previously described, using a continuous perfusion of the following sequence of buffers: (i) Hepes phosphate buffered ringers solution (HPBR) containing 10 mM Hepes pH 7.4, 145 mM NaCl, 5 mM KCl, 1.2 mM MgSO4, 1.2 mM Ca-gluconate, 2.4 mM K2HPO4, 0.4 mM KH2PO4, (ii) HPBR supplemented with 100 mm amiloride, (iii) HPBR (Cl− free) containing 10 mM Hepes pH 7.4, 145 mM Na-gluconate, 5 mM K-gluconate, 1.2 mM MgSO4, 1.2 mM Ca-gluconate, 2.4 mM K2βPO4, 0.4 mM KH2βO4, 100 mM amiloride, (iv) HPBR (Cl− free) supplemented with 100 mM amiloride, 200 mM 8-cpt-cAMP, 10 mM forskolin, (v) HPBR (Cl− free) supplemented with 100 mM amiloride, 200 mM 8-cpt-cAMP, 10 mM forskolin, 100 mM UTP, and (vi) HPBR. These conditions are used to create a chemical driving force by which changes in Cl− permeability can be assessed following cAMP and UTP agonist stimulation. Measurements of UTP-simulated Cl− secretion through the Ca+-activated Cl− channel are used to control for the integrity of the epithelium. Millivolt recordings are taken by computer-assisted data link every five seconds.

Changes in the transepithelial PD following cAMP stimulation are proportional to the extent of CFTR correction while changes in transepithelial PD induced by UTP indicate Cl− secretion through the Ca+-activated chloride channel (serve as a control for the electrical integrity of the SAE).

Following completion of functional studies, xenograft airways are assayed to confirm PTM mRNA expression using in situ hybridization and a probe specific for the PTM, or a universal probe to sequences within the 3′UTR of the trans-spliced CFTR transcripts. Localization of CFTR with regard to the apical surface of airway cells is examined by immunofluorescence microscopy using antibodies that detect either CFTR protein or a tag incorporated in the PTM delivered sequence. This allows for a direct assessment of stem cell reconstitution and is compared to that found with full length CFTR cDNA retroviral vectors. Tissues will also be evaluated by RT-PCR and Western blot analysis for mRNA and protein repair.

Successful trans-splicing using the PTM based vectors for correcting endogenous CFTR mRNA should result in functional changes in chloride permeability in response to cAMP agonists. If the efficiency of trans-splicing is 100%, based on observations with adenoviral complementation, as little as 10% transduction of xenografts with retroviral based PTMs should allow for full functional correction. To increase efficiency airway cells may be transduced with multiple PTM constructs targeting different CFTR intron sites which can repair by trans-splicing in the appropriate exonic sequences. Alternatively, it is possible to generate mutifunctional retroviral vectors encoding more than one CF repair PTM under different promoters.

The present invention is not to be limited in scope by the specific embodiments described herein. Indeed, various modifications of the invention in addition to those described herein will become apparent to those skilled in the art from the foregoing description and accompanying Figures. Such modifications are intended to fall within the scope of the appended claims. Various references are cited herein, the disclosure of which are incorporated by reference in their entireties.

We claim:

1. A stem cell comprising a nucleic acid molecule wherein said nucleic acid molecule comprises:
a) one or more target binding domains that target binding of the nucleic acid molecule to pre-mRNA expressed within the stem cell or a differentiating stem cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the stem cell or differentiating stem cell.

2. A stem cell comprising a nucleic acid molecule wherein said nucleic acid molecule comprises:
a) one or more target binding domains that target binding of the nucleic acid molecule to a pre-mRNA expressed within the stem cell or a differentiating stem cell;
b) a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the stem cell or the differentiating stem cell.

3. A stem cell comprising a nucleic acid molecule wherein said nucleic acid molecule comprises:
a) one or more target binding domains that target binding of the nucleic acid molecule to a pre-mRNA expressed within the stem cell or a differentiating stem cell;
b) a 5′ splice site;
c) a spacer region that separates the 5′ splice site from the target binding domain; and
d) a nucleotide sequence to be trans-spliced to the target pre-mRNA;
wherein said nucleic acid molecule is recognized by nuclear splicing components within the stem cell or a differentiating stem cell.

4. The stem cell of claim 1 or 2 wherein the nucleic acid molecule further comprises a 5′ donor site.

5. The stem cell of claim 1 or 2 wherein the 3′ splice region further comprises a pyrimidine tract.

6. The stem cell of claim 1, 2 or 3 wherein said nucleic acid molecule further comprises a safety sequence comprising one or more complementary sequences that bind to one or both sides of the 5′ splice site.

7. The stem cell of claim 1, 2 or 3 wherein the nucleic acid molecule further comprises a safety nucleotide sequence comprising one or more complementary sequences that bind to one or more sides of the 3′ splice region.

8. The stem cell of claim 1 or 2 wherein the binding of the nucleic acid molecule to the target pre-mRNA is mediated by complementary, triple helix formation, or protein-nucleic acid interaction.

9. The stem cell of claim 1 or 2 wherein trans-splicing of the nucleotide sequence to the target pre mRNA results in correction of a genetic disorder.

10. A stem cell comprising a recombinant vector wherein said vector expresses a nucleic acid molecule comprising:
a) one or more target binding domains that target binding of the nucleic acid molecule to a pre-mRNA expressed within the stem cell or a differentiating stem cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the stem cell or differentiating stem cell.

11. A stem cell comprising a recombinant vector wherein said vector expresses a nucleic acid molecule comprising:
a) one or more target binding domains that target binding of the nucleic acid molecule to a pre-mRNA expressed within the stem cell or a differentiating stem cell;
b) a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the stem cell or differentiating stem cell.

12. A stem cell comprising a recombinant vector wherein said vector expresses a nucleic acid molecule comprising:
a) one or more target binding domains that target binding of the nucleic acid molecule to pre-mRNA expressed within the stem cell or a differentiating stem cell;
b) a 5′ splice site;
c) a spacer region that separates the 5′ splice site from the target binding domain; and
d) a nucleotide sequence to be trans-spliced to the target pre-mRNA;
wherein said nucleic acid molecule is recognized by nuclear splicing components within the stem cell or differentiating stem cell.

13. The stem cell of claim 10 or 11 wherein the nucleic acid molecule further comprises a 5′ donor site.

14. The stem cell of claim 10 or 11 wherein the 3′ splice region further comprises a pyrimidine tract.

15. The stem cell of claim 10, 11, or 12 wherein the nucleic acid molecule further comprises a safety nucleotide sequence comprising one or more complementary sequences that bind to one or more sides of the 3′ splice region and/or 5′ splice site.

16. A method of producing a chimeric RNA molecule in a stem cell or differentiating stem cell comprising:
contacting a pre-mRNA expressed in the cell with a nucleic acid molecule recognized by nuclear splicing components wherein said nucleic acid molecule comprises:
a) one or more target binding domains that target binding of the nucleic acid molecule to a pre-mRNA expressed within the stem cell or differentiating stem cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA;
under conditions in which a portion of the nucleic acid molecule is trans-spliced to a portion of the target pre-mRNA to form a chimeric RNA within the stem cell or differentiating stem cell.

17. A method of producing a chimeric RNA molecule in a stem cell or differentiating stem cell comprising:
contacting a pre-mRNA expressed in the stem cell or differentiating stem cell with a nucleic acid molecule recognized by nuclear splicing components wherein said nucleic acid molecule comprises:
a) one or more target binding domains that target binding of the nucleic acid molecule to pre-mRNA expressed within the cell;
b) a 3′ splice acceptor site;
c) a spacer region that separates the 3′ splice region from the target binding domain; and
d) a nucleotide sequence to be trans-spliced to the target pre-mRNA wherein trans-splicing of said nucleotide sequence results in correction of a genetic defect;
under conditions in which a portion of the nucleic acid molecule is trans-spliced to a portion of the target pre-mRNA to form a chimeric RNA within the stem cell or differentiating stem cell.

18. A method of producing a chimeric RNA molecule in a stem cell or differentiating stem cell comprising:
contacting a target pre-mRNA expressed within the stem cell or differentiating stem cell with a nucleic acid molecule recognized by nuclear splicing components wherein said nucleic acid molecule comprises:
a) one or more target binding domains that target binding of the nucleic acid molecule to a pre-mRNA expressed within the stem cell or differentiating stem cell;
b) a 5′ splice site;
c) a spacer region that separates the 5′ splice site from the target binding domain; and
d) a nucleotide sequence to be trans-spliced to the target pre-mRNA wherein said trans-splicing results in correction of a genetic defect; and
wherein said nucleic acid molecule is recognized by nuclear splicing components within the stem cell or differentiating stem cell.

19. The method of claim 16 or 17 wherein the nucleic acid molecule further comprises a 5′ donor site.

20. The method of claim 16 or 17 wherein the 3′ splice region further comprises a pyrimidine tract.

21. The method of claim 16, 17 or 18 wherein the nucleic acid molecule further comprises a safety nucleotide sequence comprising one or more complementary sequences that bind to one or more sides of the 3′ splice region and/or 5′ splice region.

22. The method of claim 16 wherein trans-splicing of the nucleotide sequence to the target pre mRNA results in correction of a genetic disorder.

23. A nucleic acid molecule comprising:
a) one or more target binding domains that target binding of the nucleic acid molecule to a pre-mRNA expressed within a stem cell or differentiating stem cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the stem cell or differentiating stem cell.

24. A nucleic acid molecule comprising:
a) one or more target binding domains that target binding of the nucleic acid molecule to a pre-mRNA expressed within a stem cell or differentiating stem cell;
b) a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the stem cell or differentiating stem cell.

25. A nucleic acid molecule comprising:
a) one or more target binding domains that target binding of the nucleic acid molecule to a pre-mRNA expressed within a stem cell or differentiating stem cell;
b) a 5′ splice site;
c) a spacer region that separates the 5′ splice site from the target binding domain; and
d) a nucleotide sequence to be trans-spliced to the target pre-mRNA;
wherein said nucleic acid molecule is recognized by nuclear splicing components within the stem cell or differentiating stem cell.

26. The nucleic acid molecule of claim 23 or 24 wherein the nucleic acid molecule further comprises a 5′ donor site.

27. The nucleic acid molecule of claim 23 or 24 wherein the 3′ splice region further comprises a pyrimidine tract.

28. The nucleic acid molecule of claim 23, 24, 25 wherein the nucleic acid molecule further comprises a safety nucleotide sequence comprising one or more complementary sequences that bind to one or more sides of the 3′ splice region and/or a 5′ splice site.

29. The nucleic acid molecule of claim 23 wherein the binding of the nucleic acid molecule to the target pre-mRNA is mediated by complementary, triple helix formation, or protein-nucleic acid interaction.

30. The nucleic acid molecule of claim 23 wherein trans-splicing of the nucleotide sequences to the target pre mRNA results in correction of a genetic defect.

31. A eukaryotic expression vector wherein said vector expresses a nucleic acid molecule comprising:
a) one or more target binding domains that target binding of the nucleic acid molecule to a pre-mRNA expressed within a stem cell or differentiating stem cell;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the stem cell or differentiating stem cell.

32. A eukaryotic expression vector wherein said vector expresses a nucleic acid molecule comprising:
a) one or more target binding domains that target binding of the nucleic acid molecule to a pre-mRNA expressed within a stem cell or differentiating stem cell;
b) a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the stem cell or differentiating stem cell.

33. A eukaryotic expression vector wherein said vector expresses a nucleic acid molecule comprising:
a) one or more target binding domains that target binding of the nucleic acid molecule to a pre-mRNA expressed within a stem cell or differentiating stem cell;
b) a 5′ splice site;
c) a spacer region that separates the 5′ splice site from the target binding domain; and
d) a nucleotide sequence to be trans-spliced to the target pre-mRNA;
wherein said nucleic acid molecule is recognized by nuclear splicing components within the stem cell or differentiating stem cell.

34. The vector of claim 31 wherein the nucleic acid molecule further comprises a 5′ donor site.

35. The vector of claim 31 wherein the nucleic acid molecule further comprises a pyrimidine tract.

36. The vector of claim 31, 32, or 33 wherein the nucleic acid molecule further comprises a safety nucleotide sequence comprising one or more complementary sequences that bind to one or more sides of the 3′ splice region.

37. The vector of claim 31, 32 or 33 wherein said vector is a viral vector.

38. The vector of claim 31, 32, or 33 wherein expression of the nucleic acid molecule is controlled by a mammalian specific promoter.

39. A composition comprising a physiologically acceptable carrier and a nucleic acid molecule according to any of claims 23-30.

40. A method for correcting a genetic defect in a subject comprising administering to said subject a nucleic acid molecule comprising:
a) one or more target binding domains that target binding of the nucleic acid molecule to a pre-mRNA expressed within a cell wherein said pre-mRNA is encoded by a gene containing a genetic defect; and
b) a nucleotide sequence to be trans-spliced to the target pre-mRNA wherein said trans-splicing results in correction of the genetic defect; and
wherein said nucleic acid molecule is recognized by nuclear splicing components within the cell.
